(19) United States
(12) Patent Application Publication
(10) Pub. No.: US 2021/0315488 A1
(43) Pub. Date: Oct. 14, 2021

(54) ANALYSING SYMMETRY OF LIMB FUNCTION

(71) Applicant: Opum Technologies Limited, Auckland (NZ)

(72) Inventor: Andrew John McDaid, Auckland (NZ)

(21) Appl. No.: 17/271,584

(22) PCT Filed: Aug. 28, 2019
(86) PCT No.: PCT/NZ2019/050111

§ 371 Date: Feb. 25, 2021

(30) Foreign Application Priority Data
Aug. 28, 2018 (NZ) 745770

(51) Int. Cl. A61B 5/11; A61B 5/00

(52) U.S. Cl. CPC A61B 5/1121; A61B 5/1112; A61B 5/112; A61B 5/6812; A61B 2562/0261; A61B 5/742; A61B 2505/09; A61B 2562/0219; A61B 2562/0223; A61B 5/6811

(57) ABSTRACT

Systems and methods for analysis of symmetry between sides of a body. A wearable device includes a body mounting portion structured and arranged to, in use, be mounted to one or more parts of a body of a patient on one side of the body. The wearable device includes at least one sensor configured to output a signal indicative of at least one physiological parameter from a side of the body to which the wearable device is mounted. At least one processor is configured to receive at least one physiological parameter from the wearable device while mounted to a first side of the body, and at least one physiological parameter from the wearable device while mounted to a second side of the body. An indicator of symmetry between the first side and the second side of the body is determined based at least in part on the at least one physiological parameter from the first side of the body and the at least one physiological parameter from the second side of the body.

### 1 STATEMENT OF CORRESPONDING APPLICATIONS

This application is based on the provisional specification filed in relation to New Zealand Patent Application No. 745770, the entire contents of which are incorporated herein by reference.

### 2 FIELD OF TECHNOLOGY

The present technology is directed to methods, devices, and systems for analysing bilateral symmetry of a human or animal patient—more particularly bilateral symmetry between limbs of the patient.

### 3 BACKGROUND TO THE TECHNOLOGY

Symmetry of limb function, particularly where impairment of a limb is present, is an important biomarker for injury recovery (e.g. from a stroke, or musculoskeletal injury such as knee ACL tear), disease diagnosis (e.g. hemiparesis in cerebral palsy), as well as optimised human performance and injury prevention in sports (e.g. running).

It is known to use clinical measures from functional evaluations on each limb (e.g. hop tests on each leg) to assess symmetry. However, these measures are often subjective. Further, such measures need to be manually digitalised for logging and tracking—increasing the potential for errors in data entry, and/or failure to enter the data at all.

Objective measures such as electrogoniometers, optical motion capture and wearable sensor networks—typically including a plurality of inertial measurement units (IMUs)—are known for determining both limb kinematics (describing motion of the limb or a joint thereof) and kinetics (describing moments and loads associated with motion of the limb or joint thereof) to assess symmetry.

One problem with existing systems is their expense and therefore availability for use. Wearable sensor networks are also complex and time consuming to customise—typically requiring assistance from a trained user to attach and configure the system in order to obtain accurate data. This can impact the ease of data collection and therefore tracking of symmetry (particularly in remote and/or rural settings), and generally be inconvenient to the patient.

It is an object of aspects of the technology to provide a system, device and/or method for performing analysis of symmetry of limb function to overcome or ameliorate problems with existing systems, devices and methods. Alternatively, it is an object to provide an improved system, device and/or method for analysing symmetry of limb function. Alternatively, it is an object to at least provide the public with a useful choice.

### 4 SUMMARY OF THE TECHNOLOGY

Aspects of the present disclosure are directed towards providing means for analysis of symmetry between sides of a body of a patient, particularly for use in the rehabilitation, support, monitoring, diagnosis or prevention of afflictions associated with asymmetry, or in improving performance and injury prevention.

It is envisaged that aspects of the present disclosure may have particular application to analysis of symmetry of limbs of the patient. Aspects of the present disclosure may be described herein with reference to the arms and/or legs of the patient. It should be appreciated that such references are intended to encompass musculoskeletal features of the respective limbs in their entirety, rather than be formal anatomical definitions.

One aspect of the present disclosure is a wearable device. Another aspect is a system including the wearable device and at least one processing device. Another aspect is methods of analysis of symmetry using the wearable device and/or system.

According to one aspect of the present disclosure there is provided a system for analysis of symmetry between sides of a body, the system including a wearable device, the wearable device including: a body mounting portion structured and arranged to, in use, be mounted to one or more parts of a body of a patient on one side of the body; and at least one sensor configured to output a signal indicative of at least one physiological parameter from a side of the body to which the wearable device is mounted; at least one processor configured to: receive the signal indicative of the at least one physiological parameter from the side of the body; and determine an indicator of symmetry between both sides of the body based at least in part on the at least one physiological parameter from the side of the body.

According to one aspect of the present disclosure there is provided a method of analysing symmetry between sides of a body, the method including: mounting a wearable device to one or more parts of a body of a patient on a first side of the body, the wearable device including at least one sensor configured to output a signal indicative of at least one physiological parameter from the side of the body to which the wearable device is mounted; determining an indicator of symmetry between both sides of the body based at least in part on the at least one physiological parameter from the first side of the body.

Reference to an indicator of symmetry should be understood to mean a measure by which bilateral symmetry of a patient, particularly symmetry between function of limbs or a portion thereof, may be assessed. A variety of metrics are known in the art, based on comparison of one or more physiological parameters between sides of the patient. More particularly, it is envisaged that assessment of symmetry of limbs may be assessed based on biomechanical parameters. For example, symmetry may be analysed in terms of targeted kinematic and/or kinetic parameters (for example, range of motion of a joint), or characteristics of an activity (for example, gait parameters such as step length, cadence, peak joint angles, and/or stance time).

By way of example, it is envisaged that for all joints the characteristics for comparison may include one or more of: range of motion, joint speed, strength, and fatigue. By way of example in relation to assessment of symmetry between knees, comparison may be made between one or more of: joint load and moment during gait events, varus and/or valgus angles during an activity such as squats, or symmetry of a task (e.g. distance in a hop test performed on each leg). It is envisaged that symmetry of upper limbs may be based on an assessment of coordination, and smoothness of joint motion to perform a task (e.g. reach and grasp tasks). It should be appreciated that these are discussed by way of exemplification of embodiments of the present disclosure, and are not intended to be limiting to all embodiments.

In an exemplary embodiment, a determination may be made as to symmetry between the first side and the second side based on the indicator of symmetry. In an exemplary embodiment, determination of symmetry may include comparison of the indicator of symmetry to a threshold value. It is envisaged that the threshold value assist with accounting for variation introduced, for example, through misalignment of the wearable device(s) or natural variation in the physiology of users. It will be appreciated that the threshold may vary for one or more of: different types of wearable device and/or mechanism of measurement, the physiological parameter being measured (e.g. the range of motion for thigh movement will be expected to be different to that for knee movement, and as such the threshold may be different for an indicator of symmetry for thigh range of motion in comparison with one for knee range of motion), type of assessment (e.g. walking vs running), injury type, injury stage, level of symmetry that can be tolerated (e.g. before return to sport), or a clinician's personal opinion as to acceptable levels of symmetry. In exemplary embodiments the threshold value may be a proportion or a percentage of an expected value for the physiological parameter.

In exemplary embodiments, the comparison may be binary—i.e. if the indicator is above the threshold, a determination may be made that the indicator is not symmetrical. In exemplary embodiments, the comparison may be relative—i.e. a relative categorisation of symmetry may be ascribed such as very/fairly/poorly symmetric.

In an exemplary embodiment, the wearable device may be an orthosis or exoskeleton. The inventor believes that the use of such a device—inherently designed to provide therapy, assessment and/or assistance to the affected side of the body—to also assist with analysis of symmetry may be beneficial. In particular, the body mounting portion of such devices may already be configured or adapted to an individual patient's requirements. Further, this may avoid the need to provide a specialised wearable device in addition to an orthosis or exoskeleton, and the process of the patient transitioning between them (i.e. taking off orthosis, putting wearable device on, taking wearable device off, and putting orthosis back on). Another potential benefit may be the ability to collect data during everyday activities, where the patient is expected to already be wearing (and be familiar with) the device in a more natural environment than during an isolated clinical evaluation. For example, characteristics of gait may be collected throughout the patient's day.

It is envisaged that embodiments of the present disclosure may have particular application to the wearable device being a knee orthosis or exoskeleton—although it should be appreciated that this is not intended to be limiting, and the wearable device may be configured to be mounted to other parts of the body. Further, it is also envisaged that in exemplary embodiments the wearable device may not be an orthosis or exoskeleton.

In an exemplary embodiment, the wearable device may be a dedicated sensing device—i.e. having the primary purpose of obtaining the variable(s) indicative of the at least one metric of symmetry. For example, the wearable device may be a wearable wireless motion tracker such as the “MTw Awinda Wireless Motion Tracker” by Xsens Technologies B.V. (https://www.xsens.com). As a further example, the wearable device may be a smart insole to inserted into footwear worn by the user.

In an exemplary embodiment, the wearable device may be an intelligent user device, whether designed to be worn in general use (for example, a smart watch or fitness/activity tracker), or capable of being worn in combination with a body mounting accessory (for example, a smart phone held by a band or straps, or inserted into a pocket of clothing—particularly compression clothing restricting movement relative to the patient's body). Such devices are known to include sensors which might be utilised for the purposes of exemplary embodiments of the present disclosure—for example an inertial measurement unit (IMU) or functional equivalent thereof. As a further example, the wearable device may be an item of smart clothing or footwear having integrated sensor(s).

In an exemplary embodiment, the wearable device sensor may be removably attached to the body mounting portion. It is envisaged that this may be particularly applicable to exemplary embodiments in which a single wearable device is to be mounted to body parts on opposing sides of the patient sequentially (e.g. worn on the left limb, and then worn on the right limb)—as will be discussed further below. It is envisaged that the wearable device sensor may be switched between sides of the body mounting portion when worn on the contralateral limb—i.e. the sensor and body mounting portion may be configured such that the sensor may be selectively attached to either side of the body mounting portion. By way of example, in the case of a knee brace it may be desirable for the sensor to be located on the outside of the respective legs in order that data collected for the respective legs is mirrored.

Where the wearable device is an orthosis or exoskeleton, it is envisaged that the requirement to align the device (and therefore sensor) relative to the patient's body may be reduced—such devices being designed to be easily aligned in use. However, it should be appreciated that a variety of means may be utilised to improve alignment (e.g. visual aids, or a calibration process) or account for misalignment during processing (e.g. basing analysis on averages of multiple measurements, or using multiple sensors on a single joint to provide redundancy to overcome misalignment). Further, it is envisaged that for some sensor types (e.g. inertial measuring units measuring acceleration in multiple directions), alignment is not an important factor.

It should be appreciated that the at least one sensor of the wearable device may be configured to measure any physiological variable(s) suitable for use in the analysis of symmetry of the target body part(s). More particularly, it is envisaged that the at least one sensor may be configured to measure biomechanical parameters, especially kinetic and/or kinematic parameters of the patient, or variables from which these may be derived. As referred to herein, kinetics (often called dynamics in the field of physiology) relate to the forces and torques that cause motion. Measurements can include internal musculoskeletal dynamics such as muscle forces, and joint loads and torques as well as externally acting forces and torques (such as foot ground reaction forces when walking, or weight when lifting a load). Kinematics relate to motion without reference to force(s) and torque(s) that cause the motion.

By way of example, the one or more sensors may include one or more of: motion and/or orientation sensors (for example one or more of accelerometers and gyroscopes), including integrated devices such as an inertial measuring unit (IMU); angular displacement sensors (for example incremental sensors such as rotary encoders, or absolute position sensors), including in combination to measure angular motion in multiple directions (for example, an instrumented linkage mechanism); electrogoniometers; force sensors (for example, load cells, and pressure sensors in foot insoles); and physiological sensors, for example, a electromyography (EMG) sensor, a thermometer, a heart rate sensor, a blood pressure sensor, a blood oxygen level sensor, etc.

In an exemplary embodiment the system may include at least one reference sensor, separate to the wearable device. More particularly, it is envisaged that the reference sensor may not be included in a contralateral equivalent of the wearable device. Determination of the indicator of symmetry between the sides of the body may be based at least in part on the output of the reference sensor, as will be discussed further below.

In an exemplary embodiment, the reference sensor may, in use, be mounted to a body part of the patient other than a contralateral equivalent to the body part to which the wearable device is mounted on the first side. For example, the reference sensor may be mounted to the trunk, waist, neck, or head of the patient.

In an exemplary embodiment, the reference sensor may, in use, be mounted to a body part on the contralateral side of the patient's body from the wearable device. In an exemplary embodiment, the reference sensor may, in use, be mounted to the limb of the patient's body contralateral to that on which the wearable device is mounted.

It is envisaged that in exemplary embodiments the reference sensor may be mounted to or integrated into a prosthesis or fitting (i.e. socket). In an exemplary embodiment the prothesis or fitting may be on the contralateral side of the body to the wearable device. In an exemplary embodiment the prothesis or fitting may be or include the contralateral equivalent to the part of the body to which the wearable device is mounted. For example, where the wearable device is a knee orthosis, the articulation mechanism of a prosthetic leg may be instrumented, or have a reference sensor mounted thereto.

It should be appreciated that the at least one reference sensor may be configured to measure any variable(s) suitable for use as a reference to data from the wearable device sensor for determination of symmetry. Such sensors may include those discussed above in relation to the wearable device sensor—for example, force sensors or IMU for detection of heel strike. In exemplary embodiments the reference sensor may include a sensor configured to indicate spatial, temporal, and/or spatio-temporal parameters of the patient as a whole, for example distance travelled, velocity relative to ground and/or trunk motion.

In an exemplary embodiment, the method of analysing symmetry may include: receiving a first data set collected from the sensor of the wearable device while mounted to one or more parts of a body of a patient on a first side of the body; receiving a second data set collected from the sensor of the wearable device while mounted to one or more contralateral parts of the body of the patient, wherein determining the indicator of symmetry between both sides of the body is based at least in part on analysis of the first data set and the second data set.

According to one aspect of the present disclosure there is provided a method of analysing symmetry between sides of a body, the method including: mounting a wearable device to one or more parts of a body of a patient on a first side of the body, the wearable device including at least one sensor configured to output a signal indicative of at least one physiological parameter from the side of the body to which the wearable device is mounted; recording a first data set from the at least one sensor while the patient performs at least one activity while the wearable device is mounted on the first side; mounting the wearable device to one or more parts of a body of the patient on a second side of the body contralateral to the first side; recording a second data set from the at least one sensor while the patient performs at least one activity while the wearable device is mounted on the second side; and determining the indicator of symmetry between both sides of the body based at least in part on analysis of the first data set and the second data set.

In an exemplary embodiment, the activity of the patient during collection of the first data set may be repeated for collection of the data set. For example, the activity may be one or more defined functional tasks appropriate to the target body part for assessment, such as the hop test, box and blocks test or reach and grasp tasks. However, it should be appreciated that this is not intended to be limiting to all embodiments, as it is envisaged that comparable data may be obtained by performing different activities while wearing the wearable device on the respective sides.

In exemplary embodiments, the wearable device may be physically reconfigured between use of different sides of the body—for example, repositioning of the wearable device sensor relative to the body mounting portion such that it mirrors the configuration when mounted to the other side of the body.

It is envisaged that determination of a side of the body to which the wearable device is mounted may be performed manually, or automatically. For example, a user (whether the patient or an operator) may designate a side on which the wearable device was mounted during collection of a data set. This may be performed prior to data collection or following. By way of example, the manual designation may include one or more of: operating a physical selectable device on the wearable device (e.g. one or more buttons or switches), and inputting a selection of a side via a graphical user interface.

In an exemplary embodiment, the side of the body on which the wearable device is located may be determined automatically. It is envisaged that data from the wearable device sensor may be used to do so. For example, where the wearable device is a knee orthosis, medial-lateral sway may be used to infer whether the wearable device is worn on the left or right limb. In such an embodiment, lateral rotation at the hip joint could be used during gait (for example, detected by an IMU on the knee). The knee/leg will tend to rotate outwards during the swing phase of gait (as it cannot rotate inwards without striking the other leg). As a further example, data relating to the range of motion of joint(s) may be used to infer whether the wearable device is worn on the left or right limb. By way of example, where the wearable sensor is a rotation sensor worn on a joint such as the knee or elbow, transition of the wearable of the wearable device between limb (and repositioning of the sensor) may produce mirrored outputs—i.e. a positive angle will be read during flexion on one side, and a negative angle during flexion on the contralateral side. Due to range of motion constraints on the joint, side may be inferred from the sign of the angle.

In an exemplary embodiment, the method may include performing calibration prior to collecting data—for example performing a functional task from which an indication of side may be readily derived.

In an exemplary embodiment, the method of analysing symmetry may include: receiving a first data set collected from the sensor of the wearable device while mounted to one or more parts of a body of a patient on a first side of the body; receiving a second data set collected from a reference sensor during collection of the first data set; determining the indicator of symmetry between both sides of the body based at least in part on analysis of the first data set and the second data set.

In an exemplary embodiment, the method of analysing symmetry may include: receiving a first data set collected from the sensor of the wearable device while mounted to one or more parts of a body of a patient on a first side of the body; receiving a second data set collected from the reference sensor during collection of the first data set; determining reference parameters from the first data set and the second data set; determining a physiological parameter of the contralateral parts of the body of the patient based at least in part on analysis of the reference parameters; and determining the indicator of symmetry between both sides of the body based at least in part on analysis of the estimated physiological parameter and a physiological parameter from the first data set.

The term “reference parameter” as used herein should be understood to mean a parameter indicative of activity of the patient, other than the target physiological parameter(s). By way of example, the reference parameter may be distance travelled while walking (e.g. both measured by a GPS device as the reference sensor, and estimated from step length and step count from the wearable device). Analysis of the reference parameters may allow for an estimation of the physiological parameter contralateral to that obtained from the wearable device (e.g. the difference between values of distance travelled enables an inference of the step length of the contralateral side).

In an exemplary embodiment, the method of analysing symmetry may include: receiving a first data set collected from the sensor of the wearable device while mounted to one or more parts of a body of a patient on a first side of the body; receiving a second data set collected from the reference sensor during collection of the first data set; determining a first value of a reference parameter from the first data set; determining a second value of the reference parameter from the second data set; determining a first value of a physiological parameter from the first data set; determining a second value of the physiological parameter based on analysis of at least the first value and second value of the reference parameter, wherein the second value is indicative of the physiological parameter of the contralateral parts of the body of the patient; and determining the indicator of symmetry between both sides of the body based at least in part on analysis of the first value and the second value of the physiological parameter.

In an exemplary embodiment, the method of analysing symmetry may include: receiving a first data set collected from the sensor of the wearable device while mounted to one or more parts of a body of a patient on a first side of the body; receiving a second data set collected from the reference sensor during collection of the first data set; determining a first value of a parameter from the first data set and a second value of the from the second data set; determining the indicator of symmetry between both sides of the body based at least in part on analysis of the parameters from the first data set and the second dataset.

In an exemplary embodiment, the reference sensor may be configured to measure a parameter which may be compared directly to a parameter measured by the wearable device sensor. It should be appreciated that the parameter may be measured directly, or derived or estimated from the measurables obtained by the respective sensors.

In an exemplary embodiment, the method of analysing symmetry may include: receiving a first data set collected from the sensor of the wearable device while mounted to one or more parts of a body of a patient on a first side of the body; receiving a second data set collected from the reference sensor during collection of the first data set, the second data set including reference data; determining a physiological parameter of the contralateral parts of the body of the patient based at least in part on analysis of the reference data and the first data set; and determining the indicator of symmetry between both sides of the body based at least in part on analysis of the contralateral physiological parameter, and a physiological parameter from the first data set.

By way of example, the reference data may be indicative of heel strike events (e.g. from an smart insole, or a waist worn IMU). These heel strike events may be analysed together with kinematics from the wearable device worn on one leg, to determine kinematics (and/or other parameters) of the contralateral leg. As a further example, the reference data may be used to determine over ground velocity. This may be analysed together with kinematics from the wearable device worn on one leg, to determine kinematics (and/or other parameters) of the contralateral leg. Both sets of kinematics may then be used determine the indicator of leg symmetry.

In an exemplary embodiments, determination of the indicator of symmetry and/or symmetry may be performed by one or more of at least: dedicated processor(s) of the wearable device, processor(s) of a device including a reference sensor, processor(s) of a user device (for example, a personal computing device such as a smart phone, tablet, or personal computer), and remote processing means (for example, a server or cloud computing services). The various components described herein may communicate using any suitable means known to those skilled in the art of data communication, including wired and wireless communication protocols. In exemplary embodiments, data from the wearable device and/or reference sensor may be stored locally (e.g. on removable memory device) and transferred by physical removal of the storage device. Display of a determination of symmetry, and in exemplary embodiments the underlying data, may be displayed on any suitable display device.

Further aspects of the disclosure, which should be considered in all its novel aspects, will become apparent to those skilled in the art upon reading of the following description which provides at least one example of practical application of the disclosure.

### 5 BRIEF DESCRIPTION OF THE DRAWINGS

One or more embodiments of the disclosure will be described below by way of example only, and without intending to be limiting, with reference to the following drawings, in which:

FIG. 1 is a schematic diagram showing features of a symmetry analysis system according to an aspect of the present disclosure;

FIG. 2-1 is a front perspective view of an exemplary wearable device in the form of a knee brace according to an aspect of the present disclosure;

FIG. 2-2 is a front perspective view of another exemplary wearable device in the form of a knee brace;

FIG. 3 is a front view of an exemplary wearable device in the form of an elbow brace according to an aspect of the present disclosure;

FIG. 4 illustrates transference of a wearable device between legs of a patient as part of a method of analysing symmetry according to an aspect of the present disclosure;

FIG. 5 is a flow diagram of a first exemplary method of analysing symmetry according to an aspect of the present disclosure;

FIG. 6 is a front view of a human patient demonstrating positioning of wearable devices and reference sensors according to an aspect of the present disclosure; and

FIG. 7 is a flow diagram of a second exemplary method of analysing symmetry according to an aspect of the present disclosure.

### 6 DESCRIPTION OF EXAMPLES OF THE TECHNOLOGY

It will be understood that the particular examples described herein are not intended to be limiting to all embodiments of the present technology. The various examples may share one or more common characteristics and/or features. It should be appreciated that one or more features of any one example may be combinable with one or more features of one or more other examples.

6.1 Symmetry Analysis System

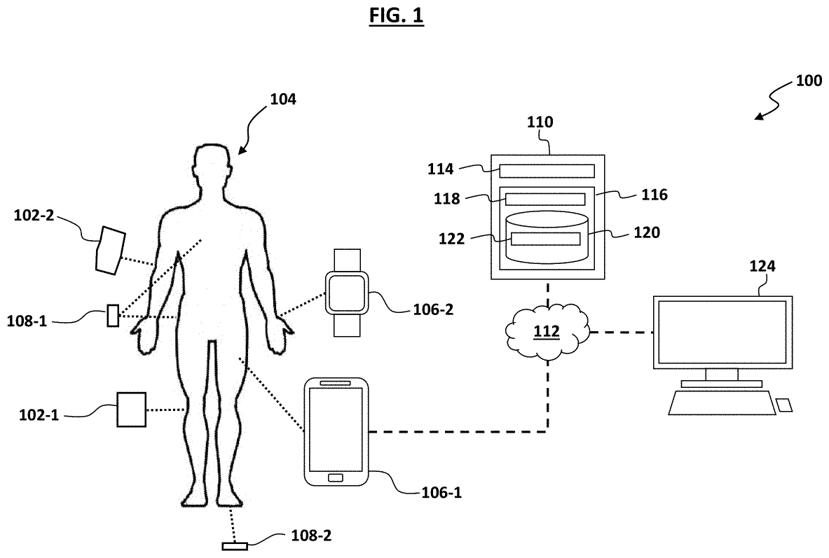

FIG. 1 is a schematic showing features of a symmetry analysis system 100 according to an embodiment of the present disclosure. The system 100 includes one or more wearable devices 102 (for example, knee orthosis 102-1 and/or elbow orthosis 102-2) configured to be mounted to a corresponding body part (s) of a body of a patient 104 in use.

The system 100 further includes one or more reference sensors, including intelligent user devices 106 (for example, smart phone 106-1 and/or smart watch 106-2) and/or dedicated reference sensor devices 108 (for example, an inertial measurement unit (IMU) 108-1 and/or smart insole 108-2).

In exemplary embodiments, data from one or more of the wearable devices 102, user devices 106, and/or reference sensor device 108 may be communicated to a remote processing service 110 via a network 112 (for example a cellular network, or another network potentially comprising various configurations and protocols including the Internet, intranets, virtual private networks, wide area networks, local networks, private networks using communication protocols proprietary to one or more companies—whether wired or wireless, or a combination thereof). For example, the smart phone 106-1 may operate an application capable of interfacing with the data management service 110.

Among other functions, the remote processing service 110 may record data, perform analysis on the received data, and report to one or more user devices. In this exemplary embodiment, the remote processing service 110 is illustrated as being implemented in a server—for example one or more dedicated server devices, or a cloud based server architecture. By way of example, cloud servers implementing the remote processing service 110 may have processing facilities represented by processors 114, memory 116, and other components typically present in such computing environments. In the exemplary embodiment illustrated the memory 116 stores information accessible by processors 114, the information including instructions 118 that may be executed by the processors 114 and data 120 that may be retrieved, manipulated or stored by the processors 114. The memory 116 may be of any suitable means known in the art, capable of storing information in a manner accessible by the processors, including a computer-readable medium, or other medium that stores data that may be read with the aid of an electronic device. The processors 114 may be any suitable device known to a person skilled in the art. Although the processors 114 and memory 116 are illustrated as being within a single unit, it should be appreciated that this is not intended to be limiting, and that the functionality of each as herein described may be performed by multiple processors and memories, that may or may not be remote from each other.

The instructions 118 may include any set of instructions suitable for execution by the processors 114. For example, the instructions 118 may be stored as computer code on the computer-readable medium. The instructions may be stored in any suitable computer language or format. Data 120 may be retrieved, stored or modified by processors 114 in accordance with the instructions 118. The data 120 may also be formatted in any suitable computer readable format. Again, while the data is illustrated as being contained at a single location, it should be appreciated that this is not intended to be limiting—the data may be stored in multiple memories or locations. The data 120 may include databases 122 storing data such as historical data associated with one or more of the one or more of the wearable devices 102, user devices 106, and/or reference sensor devices 108, and the results of analysis of same.

It should be appreciated that in exemplary embodiments the functionality of the remote processing service 110 may be realized in a local application (for example, on smart phone 106-1, or another personal computing device 124), or a combination of local and remote applications. Further, it should be appreciated that data may be transferred from one or more of the devices by other means—for example wired communication links, or transfer of storage devices such as memory cards.

The results of analysis, and/or underlying data, may be displayed on any suitable display device—for example smart phone 106-1, or computing device 124.

6.2 Knee Brace

FIG. 2-1 shows an exemplary wearable device in the form of an orthosis system particularly suited for mounting proximate a knee (not shown) of the patient (not shown)—herein referred to as first knee brace 200-1—as described in PCT application PCT/NZ2018/050085, the contents of which are incorporated herein by reference.

In this embodiment, the knee brace 200-1 includes a body mounting portion having a first brace portion 202-1 and a second brace portion 202-2. In use, the first brace portion 202-1 is mounted upwardly of the knee of the patient and the second brace portion 202-2 is mounted downwardly of the knee of the patient.

The first brace portion 202-1 and the second brace portion 202-2 are pivotably coupled via pivot assemblies 204-1 and 204-2. This makes the orthosis system 200-1 suited to use in bracing a pivoting joint of the body, such as the knee.

In other embodiments of the invention the first and second brace assemblies are moveably coupled in some other manner, for example through a sliding coupling. Such embodiments may be suitable for use in bracing an extendable part of the body, for example. In yet other embodiments, the brace of an orthosis system may be provided as a flexible sleeve, such as a continuous compression sleeve. A first portion of the sleeve is a first body mounting portion to be worn on one side of the user's joint, and a second portion of the sleeve coupled to (i.e. integrally formed with) the first portion is a second body mounting portion to be worn on an opposite side of the user's joint.

In the embodiment illustrated, modules 206-1 and 206-2 are removably coupled to the pivot assemblies 204-1 and 204-2. One or both of the modules 206-1 and 206-2 may be configured as sensing modules. While the modules 206-1 and 206-2 are illustrated as being on the sides of the patient's knee, in other embodiments the orthosis system may be configured to mount modules in other positions in relation to the body.

FIG. 2-2 shows a second knee brace 200-2 includes a body mounting portion having a first brace portion 202-1 and a second brace portion 202-2. In use, the first brace portion 202-1 is mounted upwardly of the knee of the patient and the second brace portion 202-2 is mounted downwardly of the knee of the patient. In this exemplary embodiment, the brace portions 202 are stiff arms (for example made of aluminium) having strap mounting features (for example slots 208) for positioning flexible straps (not illustrated) for mounting the knee brace 200-2 to a user.

In this embodiment, sensing module 206 is removably coupled to the pivot assembly 204 of the knee brace 200-2. The sensor module 206 includes a rotational knee movement sensor, and an IMU for sensing of thigh movement.

6.3 Elbow Brace

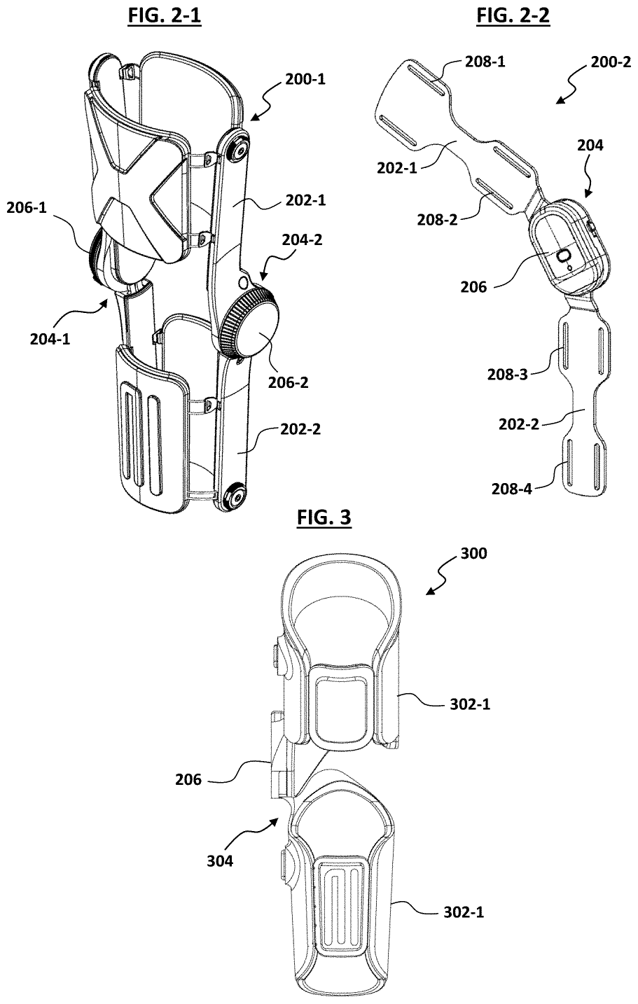

FIG. 3 shows an exemplary wearable device particularly suited for mounting proximate an elbow (not shown) of the patient (not shown)—referred to herein as elbow brace 300—as described in PCT application PCT/NZ2018/050085, the contents of which are incorporated herein by reference.

It will be noted that the body mounting portion in this embodiment comprises a first brace portion 302-1 configured to wrap around a first portion of the patient's arm. Similarly, the second brace portion 302-2 is configured to wrap around another portion of the patient's arm. The first brace portion 302-1 and the second brace portion 302-2 are pivotably coupled via pivot assembly 304-1, to which a sensor module 206 is mounted.

6.4 Sensing Module

Exemplary embodiments of the sensing module 206 comprises sensor components configured to detect, record, process and/or transmit data relating to the movement and/or rotation of the orthosis system or components thereof.

The sensor components may additionally or alternatively detect, record, process and/or transmit data pertaining to the patient's physical activity and/or physiology. This may include parameters such as joint kinematics (such as joint angle, joint velocity, joint torque, and/or joint acceleration), limb accelerations, limb rotations, limb and/or joint loads, muscle force, muscle strength, muscle velocity, electrical activity, temperature, pH, perspiration, heart rate, blood pressure and/or other bio-signals. Example sensors include rotary encoder, optical and magnetic sensors.

The sensing module 206 may comprise further components to enable the detection and recording of such data. For example, the sensing module 206 may comprise an accelerometer, gyroscope and/or magnetometers. The sensing module 206 may additionally or alternatively comprise physiological sensors, for example a thermometer, electromyography (EMG) sensor, heart rate sensor, blood pressure sensor, blood oxygen level sensor, etc.

Sensing module 206 may comprise a transmitter for transmitting data and/or signals obtained by or through the sensor components to a remote location, for example by RF, Bluetooth, Wi-Fi or any other remote communication protocol. Sensing module 206 may also comprise one or more processors configured to process the data/signals.

The sensor components may further comprise a receiver configured to receive data/signals remotely from an external source, such as external control signals. Data may be stored or received by the sensing module 206 through a physical data storage device such as a memory card, USB stick or the like.

Other sensors may be provided, comprised in or separate from a sensing module 206. For example, the wearable device 200 may also comprise a torque sensing module comprising one or more sensors for monitoring joint interaction torque between the patient and the body mounting portion. For example, such a sensor(s) may monitor relative displacement between two or more components of the device 200, for example the first and second brace portions respectively, to enable a torque sensor to sense torque between the first and second brace portions. Torque sensing may be performed when the first and second brace portions are locked, or there is some resistance between them. It should therefore be appreciated that a torque sensing module may also incorporate a locking mechanism to substantially prevent movement (e.g. rotation) between the first and second brace portions, such as described in PCT application PCT/NZ2018/050085.

A person skilled in the art will understand that a number of sensor types may be suitable for measuring characteristics of a patient's biomechanics. For example, a rotary encoder may be used to measure an angle of displacement between the first and second brace portions. Alternatively or additionally an inertial measuring unit(s) (IMU) may be attached to one or each of the first and second brace portions to measure the angle of displacement. An angle of displacement may be used to infer a resistance to motion level, by calibration of a known resistance element with respect to the amount of relative movement between the brace assemblies, or conversely a resistance measurement such as torque or force may be used to infer angle. A strain gauge may be provided to a compliant/resilient element such as a spring or elastomeric block to measure force or torque, and/or a position of a spring element may be used to indicate a resistance to motion level.

6.5 First Exemplary Method of Analysing Symmetry

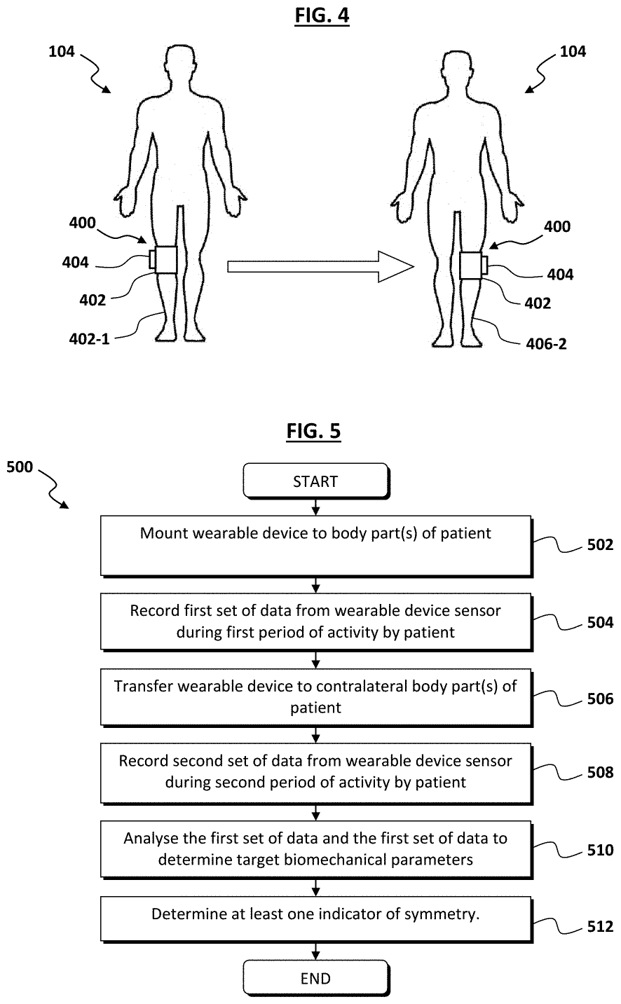

FIG. 4 and FIG. 5 illustrate a method of analysing symmetry between limbs of patient 104, demonstrated in terms of symmetry between the patient's legs. The method 500 of FIG. 5 will be described herein with reference to FIG. 4.

In a first step 502, a wearable device 400 (including body mounting portion 402 and sensor 404) is mounted to a first leg of the patient 104 (herein referred to as right leg 402-1). In a second step 504, data output from the sensor 404 during activity by the patient 104 is recorded. By way of example, the activity may be a performance of a specific task—such as a hop test, or a walk test over a designated distance—or general activity of the patient.

In a third step 506, the wearable device 400 is taken from the right leg 402-1 of the patient 104 and mounted to the contralateral leg of the patient (herein referred to as left leg 402-2). In exemplary embodiments, the wearable device 400 may be reconfigured for use with the contralateral leg. For example, where output of the sensor 404 is influenced by the side of the knee to which it is attached, the sensor 404 may be detached and applied to the other side of the body mounting portion 402.

In a fourth step 508, data output from the sensor 404 during activity by the patient 104 is recorded. The activity may correspond to that performed while the wearable device 400 was mounted to the right leg 402-1—but it is envisaged that this may not be necessary.

In exemplary embodiments, the side from which data is collected may be designated manually (for example, operating one or more buttons or switches on the wearable device 400, or inputting a selection of a side via a graphical user interface displayed on smart phone 106-1), or automatically (for example, by analysis of physiological data).

In a fifth step 510, the first set of data from the right leg 402-1 and the first set of data from the left leg 402-1 may be analysed to determine target physiological parameters for the respective legs. In a sixth step 512, at least one indicator of symmetry between the left and right sides of the patient 104 may be determined based an analysis of the physiological parameters.

By way of example, it is envisaged that for all joints the characteristics for comparison to obtain the metric(s) of symmetry may include one or more of: range of motion, joint speed, strength, and fatigue. By way of example in relation to assessment of symmetry between knees, comparison may be made between one or more of: joint load and moment during gait events, varus and/or vulgus angles during an activity such as squats, or symmetry of a task (e.g. distance in a hop test performed on each leg). It is envisaged that symmetry of upper limbs may be based on an assessment of coordination, and smoothness of joint motion to perform a task (e.g. reach and grasp tasks). It should be appreciated that these are discussed by way of exemplification of embodiments of the present disclosure and are not intended to be limiting to all embodiments.

Table 1 below includes the results from a 10 m Walk Test performed while wearing a knee brace having a knee rotational sensor for obtaining knee data, and an IMU for the thigh data (including both coronal and sagittal rotation). In this instance, the range of motion (i.e. maximum angle and minimum angle) of each parameter was obtained and compared between left and right limbs to obtain an indicator of symmetry in the form of a differential between the respective values. The indicators were then each compared against a symmetry threshold to arrive at a binary determination of whether they were symmetrical, or not. In this example, the value of the symmetry threshold is universal, however it should be appreciated that in exemplary embodiments the symmetry threshold may differ between parameters, e.g. the symmetry threshold may be a percentage of an expected range of motion of a target joint.

TABLE 1
Results of 10 m Walk Test

| | | Left | Right | Indicator | | |
|---|---|---|---|---|---|---|
| | | range of | range of | of | Symmetry | Sym- |
| | Joint | motion | motion | Symmetry | threshold | metric |
| Max | Knee | 58 | 65 | 7 | 10 | YES |
| Angle | Thigh- | 3 | 12 | 9 | 10 | YES |
| | Coronal | | | | | |
| | Thigh- | 48 | 34 | 14 | 10 | NO |
| | Sagittal | | | | | |
| Min | Knee | 0 | 0 | 0 | 10 | YES |
| Angle | Thigh- | −31 | −18 | 13 | 10 | NO |
| | Coronal | | | | | |
| | Thigh- | −32 | −24 | 8 | 10 | YES |
| | Sagittal | | | | | |

6.6 Second Exemplary Method of Analysing Symmetry

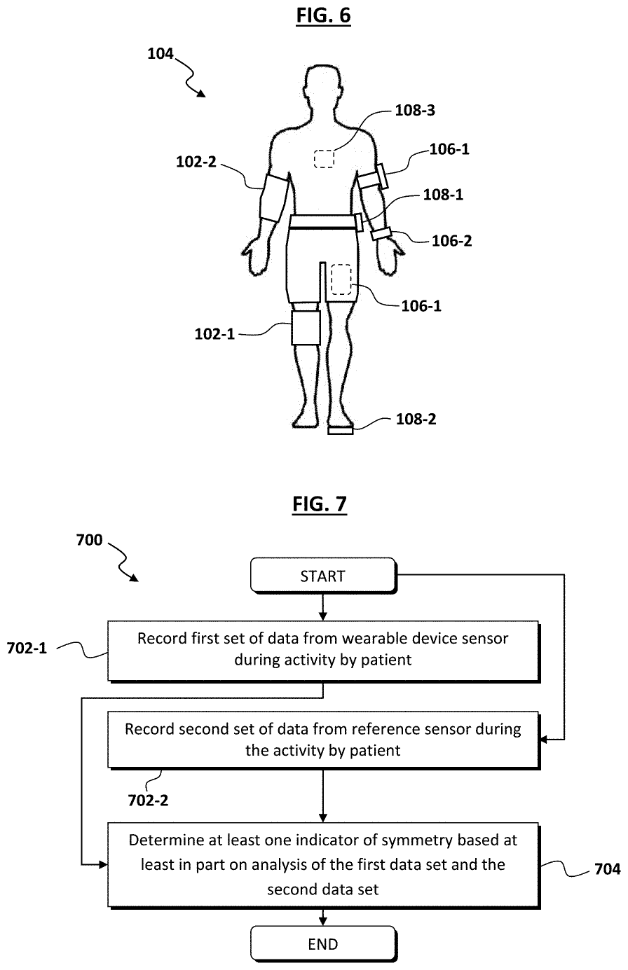

FIG. 6 and FIG. 7 illustrate another method of analysing symmetry between limbs of patient 104. The method 700 of FIG. 7 will be described herein with reference to FIG. 6.

In FIG. 6, the patient is illustrated as having a wearable device (e.g. knee orthosis 102-1 and/or elbow orthosis 102-2) mounted to a limb on a first side of their body. The patient also has at least one reference sensor mounted to their body, for example: smart phone 106-1 carried in a pocket of clothing (for example, compression shorts) or strapped to an arm or leg; smart watch 106-2; belt mounted IMU 108-1; smart insole 108-2; or wearable GPS module 108-3.

It should be appreciated that in exemplary embodiments, certain reference sensors need not be mounted on the contralateral side of the patient's body relative to the wearable device(s). Further, it should be appreciated that while multiple wearable devices and reference sensors are illustrated, all of said devices may not be worn (or used) simultaneously.

In a first step 702-1, data output from the sensor of the wearable device 102 during activity by the patient 104 is recorded. By way of example, the activity may be a performance of a specific task—such a walk test over a designated distance—or general activity of the patient.

In a second step 702-2, data output from the reference sensor during the activity by the patient 104 is recorded—i.e. from the same time period as the data collected from the wearable device 102.

In a third step 704, an indicator of symmetry between the limb on which the wearable device is mounted and the contralateral limb may be determined based at least in part on analysis of the first data set and the second data set.

In a first example, the respective data sets from the wearable device and reference sensor may include comparable parameters. For example, the patient may undergo a 10 metre walk test, with parameters (such as the distance travelled, speed, number of steps, step length, etc.) measured from both the wearable device and the reference sensor, and then compared to determine the indicator of symmetry. As an example, the indicator of symmetry may be the difference in one or more of these parameters between legs.

In a second example, the data set from the wearable device may be analysed to determine a reference parameter which may be compared with reference parameters from the reference sensor to infer a physiological parameter of the contralateral parts of the body. For example, the patient's activity may include community walking. Total distance travelled may be measured using GPS module 108-3, with the wearable device 102-1 measuring parameters such as step length and step count. The expected distance travelled based on step length of the wearable device leg may be compared with the GPS distance travelled to infer the step length of the contralateral leg. The indicator of symmetry may be the difference in step length.

In a third example, the wearable device may measure one or more physiological parameters of the first leg, and the reference sensor may measure one or more different physiological parameters. The parameters from the reference sensor, in combination with those from the wearable sensor, may be used to infer parameters for the contralateral leg equivalent to those measured by the wearable device.

For example, the reference sensor may be the belt mounted IMU 108-1, or smart insole 108-2, and detect heel strike events during activity by the patient. These may be used with kinematics measured by the wearable device for one leg to infer equivalent kinematics for the contralateral leg, from which indicators of symmetry may be determined.

In a fourth example, the wearable device may measure one or more physiological parameters of the first leg, and the reference sensor may measure one or more special-temporal parameters. The parameters from the reference sensor, in combination with those from the wearable sensor, may be used to infer parameters for the contralateral leg equivalent to those measured by the wearable device.

For example, the reference sensor may be used to determine over ground velocity during activity by the patient. These may be used with kinematics measured by the wearable device for one leg to infer equivalent kinematics for the contralateral leg, from which indicators of symmetry may be determined.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Aspects of the present technology may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present technology.

1. A method of analysing symmetry between sides of a body, the method including:
mounting a wearable device to one or more parts of a body of a patient on a first side of the body, the wearable device including at least one sensor configured to output a signal indicative of at least one physiological parameter from the side of the body to which the wearable device is mounted;
recording a first data set from the at least one sensor while the patient performs at least one activity while the wearable device is mounted on the first side;
mounting the wearable device to one or more parts of a body of the patient on a second side of the body contralateral to the first side;
recording a second data set from the at least one sensor while the patient performs at least one activity while the wearable device is mounted on the second side; and
determining an indicator of symmetry between the first side and the second side of the body based at least in part on analysis of the first data set and the second data set.

2. The method of claim 1, including determining symmetry between the first side and the second side of the body based on the indicator of symmetry.

3. The method of claim 2, wherein determining symmetry includes comparing the indicator of symmetry to a threshold value.

4. The method of claim 3, wherein determining symmetry includes a binary determination of whether the indicator of symmetry exceeds the threshold value.

5. The method of claim 3, wherein determining symmetry includes determining a relative categorisation of symmetry based on a differential between the indicator of symmetry and the threshold value.

6. The method of claim 1, wherein the indicator of symmetry is a differential between a first value of the at least one physiological parameter from the first side of the body and a second value of the at least one physiological parameter from the second side of the body.

7. The method of claim 1, wherein the at least one sensor is removably attached to a body mounting portion of the wearable device, and the method includes positioning the sensor on a first side of the body mounting portion prior to recording the first data set, and positioning the sensor on a second side of the body mounting portion prior to recording the second data set.

8. The method of claim 1, wherein the at least one sensor includes a first sensor configured to output a signal indicative of a first physiological parameter, and a second sensor configured to output a signal indicative of a second physiological parameter, and the method includes determining an indicator of symmetry for each of the first physiological parameter and the second physiological parameter.

9. The method of claim 8, wherein the wearable device is configured to be mounted to a knee of the user, and the first physiological parameter is knee range of motion, and the second physiological parameter is thigh range of motion.

10. The method of claim 1, including determining the side of the body to which the first data set and the second data set relates.

11. The method of claim 10, wherein determining the side of the body to which the first data set and the second data set relates is performed automatically.

12. A system for analysis of symmetry between sides of a body, the system including:
a wearable device, the wearable device including:
a body mounting portion structured and arranged to, in use, be mounted to one or more parts of a body of a patient on one side of the body; and
at least one sensor configured to output a signal indicative of at least one physiological parameter from a side of the body to which the wearable device is mounted;
at least one processor configured to:
receive at least one physiological parameter from the wearable device while mounted to a first side of the body;
receive at least one physiological parameter from the wearable device while mounted to a second side of the body;
determine an indicator of symmetry between the first side and the second side of the body based at least in part on the at least one physiological parameter from the first side of the body and the at least one physiological parameter from the second side of the body.

13. The claim of claim 12, wherein the wearable device is one of an orthosis or an exoskeleton.

14. The system of claim 12, wherein the wearable device includes a body mounting portion, and the at least one sensor is configured to be removably attached to the body mounting portion.

15. The system of claim 14, wherein the body mounting portion has a first side and a second side, and the at least one sensor is configured to be selectively attached to the first side or the second side of the body mounting portion.

16. The system of claim 12, wherein the at least one sensor includes a first sensor configured to output a signal indicative of a first physiological parameter, and a second sensor configured to output a signal indicative of a second physiological parameter.

17. The system of claim 12, wherein the at least one processor is configured to determine symmetry between the first side and the second side of the body based on the indicator of symmetry.

18. The system of claim 17, wherein the system includes a display device configured to display at least one of the indicator of symmetry and the symmetry.
